SEQUENCE LISTING (5 sequences)

SEQ ID NO: 1 - DNA, 62 bp, Artificial Sequence
  Description of Artificial Sequence Artificially Synthesized oligo DNA
caccgtagattatcagttatcatgggtgtgctgtccccatgatgactgatagtctgcttttt

SEQ ID NO: 2 - DNA, 62 bp, Artificial Sequence
  Description of Artificial Sequence Artificially synthesized oligo DNA
caccgcttggagggtggaatactgggtgtgctgtccccggtgttccaccctccaggcttttt

SEQ ID NO: 3 - DNA, 62 bp, Artificial Sequence
  Description of Artificial Sequence Artificially Synthesized oligo DNA
caccggagggggtattcataattaggtgtgctgtccctgattatgggtaccccctccttttt

SEQ ID NO: 4 - DNA, 62 bp, Artificial Sequence
  Description of Artificial Sequence Artificially Synthesized Oligo DNA
caccgcagtacatggtcactactacgtgtgctgtccgtggtggtgaccgtgtgctgcttttt

SEQ ID NO: 5 - DNA, 62 bp, Artificial Sequence
  Description of Artificial Sequence Artificially Synthesized Oligo DNA
caccgatctagcagtactgtgctttgtgtgctgtccaaagcacggtgctgctggatcttttt

(19) United States
(12) Patent Application Publication
(10) Pub. No.: US 2005/0053583 A1
(43) Pub. Date: Mar. 10, 2005

(54) Method of inhibiting prion protein

(75) Inventors: Suehiro Sakaguchi, Nagasaki-shi (JP); Kazunari Taira, Tsukuba-shi (JP)

Correspondence Address: NIXON & VANDERHYE, PC, 1100 N GLEBE ROAD, 8TH FLOOR, ARLINGTON, VA, 22201-4714, US

(73) Assignee: Nagasaki University, National University Corporation, Nagasaki-shi (JP)

(21) Appl. No.: 10/926,011

(22) Filed: Aug. 26, 2004

(30) Foreign Application Priority Data
Aug. 29, 2003 (JP) 2003-307666

(51) Int. Cl. A01K 67/027; A61K 48/00

(52) U.S. Cl. 424/93.2; 800/15

(57) ABSTRACT

By inhibiting expression of a normal prion protein in prion-infected cells by using siRNA, it is possible to inhibit accumulation of an abnormal prion protein consequently. The inhibition of the abnormal prion protein accumulation can be applied in prevention and treatment of the prion diseases. This provides a method of inhibiting the abnormal prion protein accumulation. This method is applicable in preventing and treating the prion diseases whose effective method of treating has not been established at this moment. Further, use of this method is provided herein.

This Nonprovisional application claims priority under 35 U.S.C. §119(a) on Patent Application No. 2003/307666 filed in Japan on Aug. 29, 2003, the entire contents of which are hereby incorporated by reference.

### FIELD OF THE INVENTION

The present invention relates to a method of inhibiting the prion protein expression and use of the same.

### BACKGROUND OF THE INVENTION

“Prion disease” is a generic name of fatal and transmissible spongiform encephalophaties, which is characterized by pathological changes such as astrocyte hyperplasia, microgria hyperplasia, amyloid plaque, and neuron death associated with spongiform degeneration. A typical prion disease in animals is scrapie, which occurs in sheep and goats. Human prion diseases include Creutzfeldt-Jakob disease, Gerstmann-Sträussler-Scheinker syndrome, Kuru, fatal familial insomnia. Those diseases are infectious.

It is postulated that a pathogenic factor of the prion disease is a “prion” that is constituted of only protein but does not have a nucleic acid. All mammals have one prion protein gene. It is considered that prion protein has important roles in the nervous system. The prion protein is of two types: normal prion protein (PrP^{c}: cellular prion protein) and abnormal prion protein (PrP^{sc}: scrapie prion protein), which are same in terms of amino acid sequence but different in protein conformation. The normal prion protein is expressed in normal tissues and has no pathogenic activity, whereas the abnormal prion protein has photogenic activity. Moreover, the normal prion protein is sensitive to protease and soluble in detergents, while the abnormal prion protein is resistance to protease and insoluble in the detergents.

The following model is suggested as a hypothesis for mechanism of the pathogenesis of prion disease: an abnormal prion protein taken into a normal individual enters the brain thereof, and makes a contact with a normal prion protein already existing inside the brain. As a result, the normal prion protein is converted into an abnormal prion protein by some kind of post-translational modification. The abnormal prion protein thus generated by the conversion converts normal prion protein nearby. As a result, the abnormal prion proteins are accumulated inside the brain, thereby causing the prion disease. Today, this model is widely accepted.

Intensive researches have been carried out for development of effective methods of treating prion diseases. For example, researches have been carried out for a method of inhibiting the conversion from the normal prion protein to the abnormal prion protein, a method of converting the abnormal prion protein back to the normal prion protein, development of vaccines by using anti-prion protein antibodies or attenuated prion strains, and the like attempts.

On the other hand, RAN interference (RNAi) is a phenomenon that double-stranded RNA entering cells causes degradation of mRNA that is homologous to a sequence of the RNA and thereby inhibits gene expression. The researches have showed that siRNA (small interfering RNA), which is short double-stranded RNA, specifically inhibits expression of the gene that is homologous to the siRNA.

The inventors have already developed vectors for producing siRNA in mammalian cells by using a U6 promoter, which is a RNA polymerase III type promoter (Miyagishi M, Taira K. U6 promoter-driven siRNAs with four uridine 3′ overhangs efficiently suppress targeted gene expression in mammalian cells. Nat Biotechnol. 2002 May; 20(5): 497-500.)

Recently, Tilly et al. reported that expression of the normal prion protein is inhibited in prion non-infected cells by using an siRNA expression vector having the U6 promoter. (Tilly G, Chapuis J, Vilette D, Laude H, Vilotte J L. Efficient and specific down-regulation of prion protein expression by RNAi. Biochem Biophys Res Commun. 2003 Jun. 6;305(3):548-51.)

Even though intensive studies have been carried out for developing effective methods of treating prion diseases as described above, no effective treating method has been successfully developed. There is a desperate desire for effective methods of treating prion diseases, while there are a large number of reports on a novel type Creutzfeldt-Jakob disease caused by infection from the mad-cow disease (bovine spongiform encephalopathy) which is a prion disease for cattle, and on an iatrogenic prion disease caused as a result of transplantation of dura mater of an infected brain. Therefore, there is a need of a novel knowledge that is applicable in prevention and treatment of the prion disease.

According to the hypothesis of the mechanism of the pathogenesis of prion disease, it is considered that the abnormal prion protein is produced by structural conversion of the normal prion protein. Therefore, it is expected that inhibition of expression of the normal prion protein inhibits the accumulation of the abnormal prion protein.

Tilly et al. who reported the inhibition of the normal prion protein expression in the prion non-infected cells by using an siRNA expression vector having the U6 promoter. However, Tilly et al. did not report that the abnormal prion protein accumulation is inhibited by inhibiting the expression of the normal prion protein in the prion-infected cells.

### SUMMARY OF THE INVENTION

In view of the aforementioned problem, an object of the present invention is to provide a method of inhibiting accumulation of an abnormal prion protein, the method being applicable in prevention and treatment of the prion diseases, and to provide a use of the method.

The inventors of the present invention accomplished the present invention by finding out that a prion protein specific siRNA inhibits expression of a normal prion protein in prion-infected cells, and also inhibits the accumulation of the abnormal prion protein. An abnormal prion protein inhibition method of the present invention inhibits the accumulation an abnormal prion protein by inhibiting expression of a normal prion protein in prion-infected cells. As a method of inhibiting the expression of the normal prion protein, it is preferable that the expression of the normal prion protein be inhibited by using a prion protein specific siRNA. Especially, it is preferable that an siRNA expression vector (prion protein specific siRNA expression vector) be introduced into the prion-infected cells, the siRNA expression vector having a prion protein specific sequence. It is preferable that the siRNA expression vector be a virus vector. It is especially preferable that the virus vector (that is, the siRNA expression vector) be an adenovirus vector, a herpesvirus vector, or a retrovirus vector.

As to a prion protein targeted for the inhibition of the expression, it is preferable that the prion proteins be human prion proteins, domestic animal prion proteins, or laboratory animal prion proteins.

Moreover, the present invention is a method of preventing and/or treating a prion disease by using the abnormal prion protein inhibition method.

Moreover, the present invention includes an siRNA expression vector having a prion protein specific sequence. The siRNA expression vector is preferably a virus vector, especially preferably an adenovirus vector, a herpesvirus vector, or a retrovirus vector.

Further, the present invention includes a medicament for preventing and/or treating a prion disease, the medicament containing a prion protein specific siRNA or an siRNA expression vector having a prion protein specific sequence. Further, the present invention includes a kit for use in prion research, containing the siRNA expression vector.

For a fuller understanding of the nature and advantages of the invention, reference should be made to the ensuing detailed description taken in conjunction with the accompanying drawings.

### BRIEF DESCRIPTION OF THE DRAWINGS

FIG. 1 is a schematic view showing target areas of 5 kinds of prion protein specific siRNAs.

FIG. 2 is a schematic view showing a construction of siRNA expression vector having a prion protein specific sequence.

FIG. 3 is a view showing nucleotide sequence of oligo DNA used for constructing the 5 kinds of siRNA expression vector having a prion protein specific sequence.

FIG. 4 is a western blotting image showing a result of examination of an effect of inhibiting abnormal prion protein accumulation in Chandler prion strain infected cells.

FIG. 5 is a western blotting image showing a result of examination of an effect of inhibiting the abnormal prion protein accumulation by changing a DNA amount of siRNA (No.1) expression vector having a prion protein specific sequence in Chandler prion strain infected cells. .

FIG. 6 is a western blotting image showing a result of examination of an effect of inhibiting the abnormal prion protein accumulation by changing time after introducing, in Chandler prion strain infected cells, the siRNA (No.1) expression vector having a prion protein specific sequence.

FIG. 7 is a western blotting image showing a result of an examination of an effect of inhibiting the abnormal prion protein accumulation in Fukuoka-1 prion strain infected cells.

FIG. 8 is a western blotting image showing a result of an examination of an effect of inhibiting the abnormal prion protein accumulation in 22L prion strain infected cells.

### DESCRIPTION OF THE EMBODIMENTS

(1) Method of Inhibiting Abnormal Prion Protein

A method of the present invention for inhibiting abnormal prion protein (hereinafter, may be referred to as the present inhibition method) is a method of inhibiting accumulation of an abnormal prion protein by inhibiting expression of a normal prion protein in prion-infected cells. As a result of the inhibition of the normal prion protein expression in the prion-infected cells, accumulation of the abnormal prion protein is inhibited. There is no particular limitation in terms of a method of inhibiting the expression of the normal prion protein, provided that the method can inhibit the expression of the normal prion protein in the prion-infected cells. Here, the “prion” means an infectious protein, which is considered as a pathogenic factor for human Creutzfeldt-Jakob disease or the like diseases, that is, the “prion” means the abnormal prion protein. The “prion-infected cells” are persistently producing the abnormal prion protein. Moreover, the “normal prion protein” is a prion protein being expressed in a normal tissue and having no pathogenic activity. The “abnormal prion protein” is a prion protein having the same amino acid sequence as the normal prion protein but having a different protein conformation from the normal prion protein, and having a pathogenic activity.

[siRNA (small interfering RNA)]

One method of inhibiting the expression of the normal prion protein is use of siRNA (No.1) expression vector having a prion protein specific sequence, that is, an RNA interfering method. “RNA interference” is a phenomenon that double-stranded RNA entering cells causes degradation of mRNA that is homologous to a sequence of the RNA and thereby inhibits gene expression. The “siRNA” is short double-stranded RNA that can cause the RNA interference. Many studies have demonstrated that the siRNA inhibits the gene expression specifically.

A nucleotide sequence of siRNA may be designed based on a sequence of a prion protein gene of species to which the method of the present invention for inhibiting the abnormal prion protein accumulation is applied. Manuals for designing siRNA have been published in many literatures and books. The prion protein specific siRNA can be designed in accordance with the manuals. In Examples descried later, 5 kinds of siRNAs were designed based on a nucleotide sequence of a mouse prion protein gene (GenBank accession No. M13685), in order to inhibit the expression of the normal prion protein in a mouse neuroblast cell line. Targeted nucleotides were 83rd to 103rd nucleotides in No. 1, 180th to 200th nucleotides in No. 2, 370th to 390th nucleotides in No. 3, 651st to 671st nucleotides in No. 4, and 785th to 805th nucleotides in No.5.

The siRNA may be directly introduced into cells by lipofection method or the like. Moreover, a vector which is so constructed as to express siRNA in the cells (siRNA expression vector), may be introduced into cells thereby expressing the siRNA in the cells. In case the siRNA expression vector is used, the siRNA can exist in a longer time in the cells than in case where the siRNA is directly introduced into the cells. Thus, the use of the siRNA expression vector is advantageous over the direct introduction of siRNA in that the use of the siRNA expression vector gives higher efficiency in the inhibition of the gene expression. Therefore, it is preferable to inhibit the expression of the normal prion protein by introducing the siRNA expression vector into the cells.

[siRNA Expression Vector]

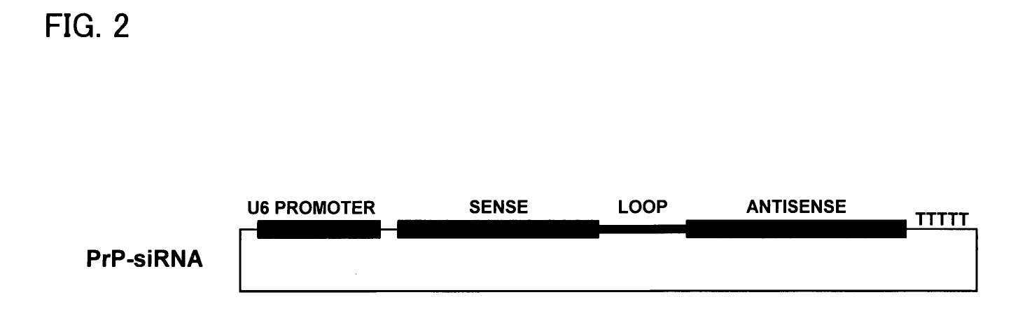

The siRNA expression vector is not particularly limited provided that the vector can express the siRNA in the cells when introduced in the cells. An example of such a vector is shown in FIG. 2: The vector has such a construction that a sense sequence, a loop sequence, an antisense sequence, and TTTTT are ligated in this order in a downstream of the U6 promoter to which a RNA polymerase binds. Moreover, the vector may have such a construction that a U6 promoter is positioned in each upstream of the sense sequence and the antisense sequence. However, the present invention is not particularly limited to the vectors having the constructions exemplified herein. Further, in terms of promoters, the present invention is not particularly limited to the U6 promoter. Any promoter to which the RNA polymerase can bind may be used. There are already-published or commercially-available plasmid vectors for expression of the siRNA. Those plasmid vectors may be suitably used. For example, the inventors have already developed vectors for producing siRNA in mammalian cells by using a U6 promoter, which is a RNA polymerase III type promoter (Miyagishi M, Taira K. U6 promoter-driven siRNAs 2ith four uridine 3′ overhangs efficiently suppress targeted gene expression in mammalian cells. Nat Biotechnol. 2002 Ma; 20(5): 497-500.) Moreover, as the commercially-available vectors, pSilencer™ 1.0-U6 siRNA Expression Vector is available from Funakoshi and the like vectors are known.

While the siRNA expression vector may be plasmid vector, a virus vector is preferable in case the vector is to introduced into a mammal. The use of the virus vector attains efficient expression of the siRNA in the cells. In case of the virus vector, a promoter for the expression of the siRNA, and a siRNA sequence are inserted in part of a virus gene. For example, an siRNA expression virus vector may be constructed by inserting, in part of the virus gene, the U6 promoter, the sense sequence, the loop sequence, the antisense sequence, and ttttt shown in FIG. 2.

There is no particular limitation in terms of the virus vector. However, it is preferable to use adenovirus vector, herpesvirus vector, or retrovirus vector. The adenovirus vector is a vector for gene therapy, and has been already used for humans. The herpesvirus vector is known for high selectivity for nerve cells, the nerve cells being target cells for the prion. The retrovirus vector has such an advantage that a gene of the retrovirus is integrated into chromosomal DNA of host cells so that the siRNA can be permanently expressed. Therefore, adenovirus vector, herpesvirus vector, or retrovirus vector is suitable as the prion protein specific siRNA vector for use in mammals including humans.

Note that the present invention includes an siRNA expression vector having a prion protein specific sequence, this siRNA expression vector being formed by inserting the prion protein specific sequence into the siRNA expression vector.

[Target Organism]

Organisms to which the method of the present invention for inhibiting the abnormal prion protein are not particularly limited as long as the organism has the prion protein gene. Among the organisms, humans, domestic animals, and laboratory animals are organisms to which the present inhibition method is preferably applicable. For humans, the present inhibition method has a possibility to be used for prevention and treatment of the prion diseases, such as Creutzfeldt-Jakob disease. The domestic animals include cattle, sheep, goats, and the like animals. As the prion diseases infecting the domestic animals, the mad-cow disease for cattle, the scrapie for sheep and goats are known. The present inhibition method has a possibility to be used as prevention and treatment of the prion diseases for the domestic animals. Moreover, the use of the present inhibition method to the laboratory animals can contribute to progress of basic study of the prion diseases. The laboratory animals include: mice, rats, hamsters, guinea pigs, rabbits, dogs, monkeys, chimpanzees, and the like.

[Method of Preventing and/or Treating Prion Diseases]

The present invention includes a method of preventing and/or treating the prion disease by using the method of inhibiting the abnormal prion protein. If the accumulation of the abnormal prion protein was inhibited by the present inhibition method, it would be possible to prevent and treat the prion disease.

(2) Medicament for Preventing and/or Treating Prion Disease

By introducing, into prion-infected cells, (a) the prion protein specific siRNA or (b) the siRNA expression vector having the prion protein specific sequence, it is possible to inhibit the expression of the normal prion protein in the cells, and consequently to inhibit the accumulation of the abnormal prion protein. Therefore, for a medicament for preventing and/or treating the prion disease, it is possible to use (a) the prion protein specific siRNA or (b) the siRNA expression vector having the prion protein specific sequence. In other words, the present invention includes a medicament for preventing and/or treating the prion disease, the medicament containing the prion protein specific siRNA, and a medicament for preventing and/or treating the prion disease, the medicament containing the siRNA expression vector, of the present invention, having the prion proteins specific sequence.

The medicament contains (a) the prion protein specific siRNA or (b) the siRNA expression vector having the prion protein specific sequence, and may contain other component. For example, the medicament may contain a buffer liquid for dissolving or suspending the siRNA or the vector.

Moreover, the present invention may be put in practice as a kit including an siRNA expression vector having a prion protein specific sequence; and a member for administering the vector. The kit may have any arrangement, provided that the kit includes the siRNA expression vector having the prion protein specific sequence; and a member for administering the vector.

The member (means) for administrating the vector is not particularly limited, and may be selected arbitrarily from well-known administrating members. Specific examples of the administrating member include a syringe, a tube, a catheter, and the like.

(3) Prion Research Kit

The siRNA expression vector, of the present invention, having the prion protein specific sequence is very useful as a tool for functional analysis of the normal prion protein or analysis of accumulation mechanism of the abnormal prion protein, because the siRNA expression vector can inhibit the expression of the normal prion protein. Therefore, a prion research kit including the siRNA expression vector having the prion protein specific sequence is applicable for promoting the prion researches. That is, the present invention includes the prion research kit (kit for use in prion research) including the siRNA expression vector having the prion protein specific sequence.

The present kit contains may have any composition, as long as the present kit contains at least the siRNA expression vector having the prion protein specific sequence. Specifically, for examples, a control vector, protenase K, a gel for electrophoresis, an agent for electrophoresis, an agent for western blotting, an antibody for prion protein detection, and/or the like may be contained.

The following describes the present invention in further details, but not limit the present invention. First, siRNAs, cells, prion stains, experiment methods used in respective Examples will be explained.

[Target Regions of siRNAs]

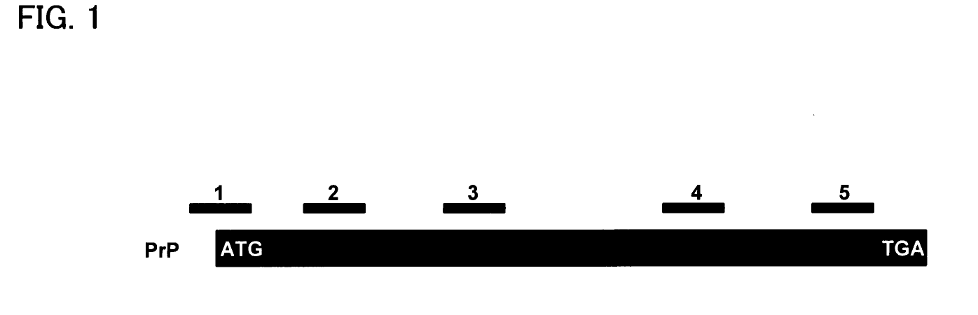

5 kinds of siRNAs were designed based on the nucleotide sequence of a mouse prion protein gene (GenBank accession No. M13685). FIG. 1 shows target region of the 5 kinds of prion protein specific siRNAs. In FIG. 1, the numerical labels 1 to 5 indicate numbering of the siRNAs. PrP indicates a prion protein. ATG indicates an initiation codon, and TGA indicates a stop codon. Targeted nucleotides are 83rd to 103rd nucleotides in No. 1, 180th to 200th nucleotides in No. 2, 370th to 390th nucleotides in No.3, 651st to 671st nucleotides in No. 4, and 785th to 805th nucleotides in No.5.

[Prion protein specific siRNA expression vector]

FIG. 2 shows a construction of a prion protein specific siRNA expression vector used in the Examples. As clearly shown in FIG. 2, the present vector is a plasmid vector, and a sense sequence and antisense sequence are connected via a loop sequence in a downstream of a U6 promoter. In a downstream of the antisense sequence, a (TTTTT) sequence is added, in order to terminate transcription performed by RNA polymerase III.

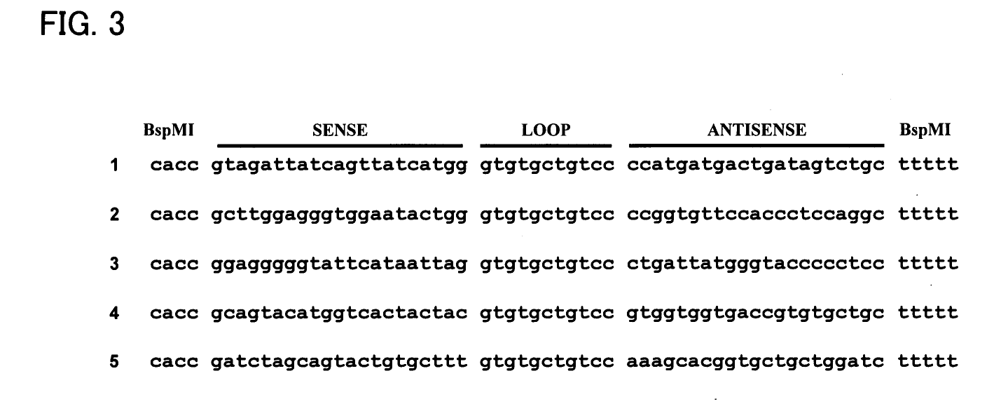

FIG. 3 shows nucleotide sequences (sequence Nos. 1 to 5) of oligo DNA used for constructing the prion protein specific siRNA expression vectors. In FIG. 3, the numerical labels 1 to 5 indicates the numbering of the siRNAs to express. BspMI is a name of a restriction endonuclease. By introducing, into targeted cells, the prion protein specific siRNA expression vector (FIG. 2) having the oligo DNA sequence shown in FIG. 3, the RNA polymerase III was bound to a region of the U6 promoter. Then, in accordance with the inserted oligo DNA sequence, RNA is synthesized in an order of from sense, loop to antisense. The thus synthesized RNA is siRNA having a hairpin construction in which a sense strand and a antisense strand form double-strands via the loop sequence.

[Cell Line Used]

N2a-58 cells that overexpressed a mouse normal prion protein was used, the N2a cells transformed by transfecting, into mouse neuroblastoma cells (N2a, ATCC CCL131), a plasmid having cDNA of a wild type mouse prion protein.

[Prion Strain Used]

Three kinds of prion strains were used in Examples, namely, Chandler prion strain, Fukukoka-1 prion strain, and 22L prion strain.

[Method of preparation of Prion-infected cells]

The prion-infected cells can be prepared by adding 1% emulsion of prion infected mouse brain to a culture medium.

[Experiment Method]

The prion protein specific siRNA expression vector or a control vector was transfected into the prion-infected cells by lipofection method. As the control vector, siRNA expression vector having a luciferase protein sequence that is irrelevant to the prion protein. After incubated for 48 hours or 96 hours, cell extracts were collected from the respective cultures. In case all the prion protein (normal prion protein and abnormal prion protein) were to be detected, the cell extracts were subjected to polyacrylamide gel electrophoresis without treating the cell extracts with protenase K. In case where only the abnormal prion protein was to be detected, the cell extracts were treated with the protenase K at 37° C. for 30 minutes, and then subjected to polyacrylamide gel electrophoresis. After that, detection of the prion protein was carried out by the western blotting method. An anti-prion protein antibody was used in the detection.

### EXAMPLE 1

### Prion Protein Inhibition Effect in Chandler Prion Strain Infected Cells

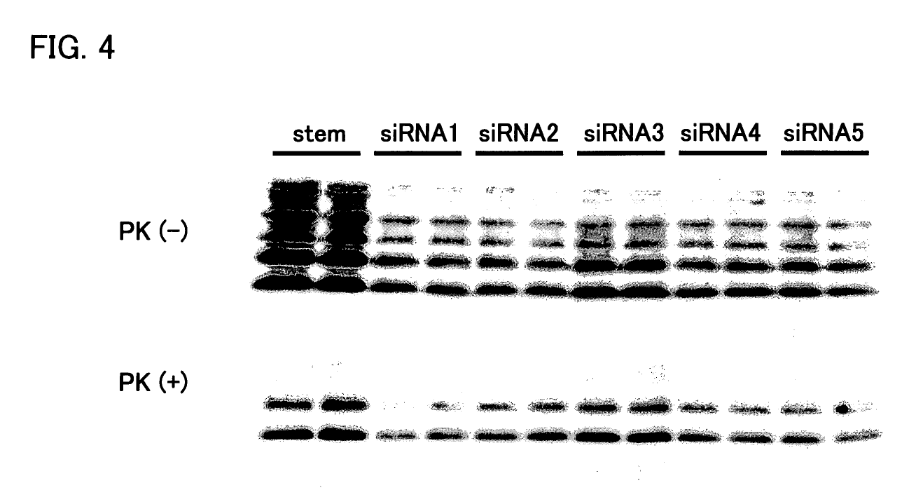

Into Chandler prion stain infected cells, the 5 kinds of prion protein specific siRNA expression vectors were transfected. After 96 hour incubation, detection of the prion proteins was carried out by the western blotting method. Results are shown in FIG. 4. In FIG. 4, stem indicates the control vector, while PK(−) and PK(+) indicate whether the protenase K treatment was carried out or not. As clearly illustrated in FIG. 4, all the 5 kinds of siRNA inhibited the production of the prion protein both in the cases of PK(−) and PK(+), compared with the control. Especially, No. 1 showed the strongest inhibition effect: an amount of the abnormal prion protein (PK(+)) detected was 20% to 30% in No. 1, compared with the control. Because of this result, only No. 1 was used as siRNA in the later Examples.

### EXAMPLE 2

### Prion Protein Inhibition Effect Dependent on Amount of Prion Protein Specific siRNA Expression Vector

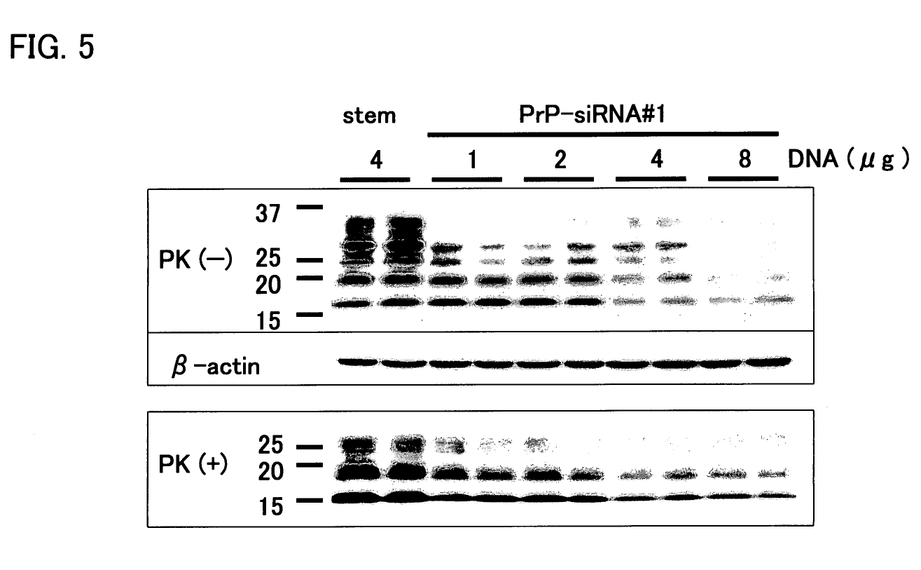

1 μg, 2 μg, 4 μg, and 8 μg of the prion protein specific siRNA (No. 1) expression vector were transfected into Chandler prion strain infected cells. An amount of the control vector was 4 μg. After 96 hours incubation, detection of the prion protein was carried out by the western blotting method. Results are shown in FIG. 5. In FIG. 5, stem indicates the control vector, while PK(−) and PK(+) indicate whether protenase K treatment was carried out or not. The numerical values on the left side (15, 20, 25, and 37) indicate molecular weights of the protein. β-actin was used as an internal control. It is clearly shown in FIG. 5 that in PK(−), the prion protein expression was inhibited in dependence on the DNA amount of the siRNA expression vector.

On the other hand, in PK(+) the accumulation of abnormal prion protein was inhibited in dependence on the DNA amount of the siRNA expression vector until 4 μg. However, there was almost no difference between 4 μg and 8 μg. This indicated that in the present experiment system, the DNA amount of 4 μg was enough for the siRNA expression vector to cause the inhibition of the abnormal prion protein accumulation.

### EXAMPLE 3

### Prion Protein Inhibition Effect Dependent on Time after Introduction of Prion Protein Specific siRNA Expression Vector

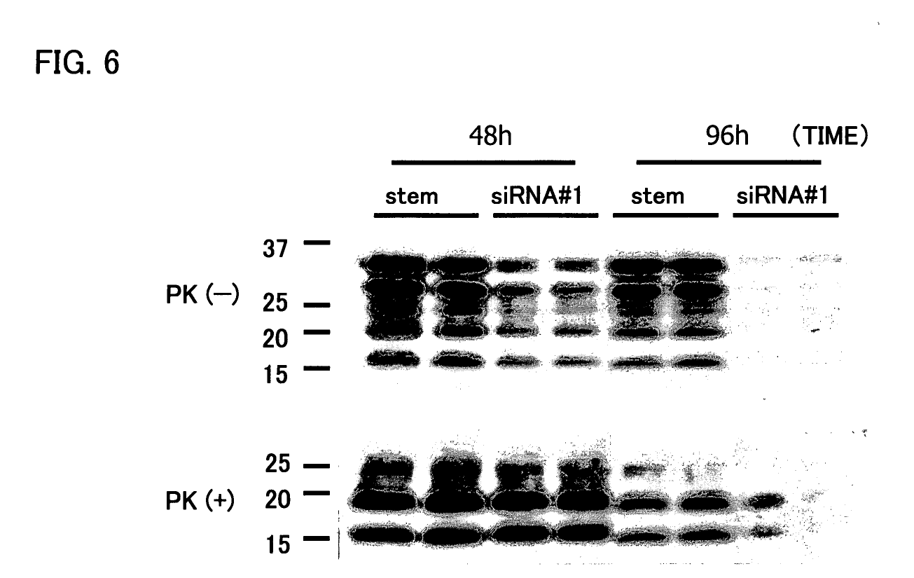

The prion protein specific siRNA (No.1) expression vector was transfected into Chandler prion strain infected cells. After 48-hour incubation and 96-hour incubation, detection of the prion protein was carried out by the western blotting method. Results are shown in FIG. 6.

In FIG. 6, stem indicates the control vector. PK(−) and PK(+) indicate whether the protenase K treatment was carried out or not. The numerical values on the left side indicate the molecular weights of the proteins. It is clearly shown in FIG. 6 that the prion protein expression was inhibited in dependence of time both in PK(−) and PK(+). However, for the 48-hour incubation, little inhibition effect was observed in PK(+), while some inhibition effect was clearly shown in PK(−). This result explains that in the present experiment system the inhibition of the abnormal prion protein accumulation requires at least 48 hours or longer and more preferably requires 96 hours or longer, after the introduction of the prion protein specific siRNA expression vector.

### EXAMPLE 4

### Prion Protein Inhibition Effect in Fukuoka-1 Prion Strain Infected Cells

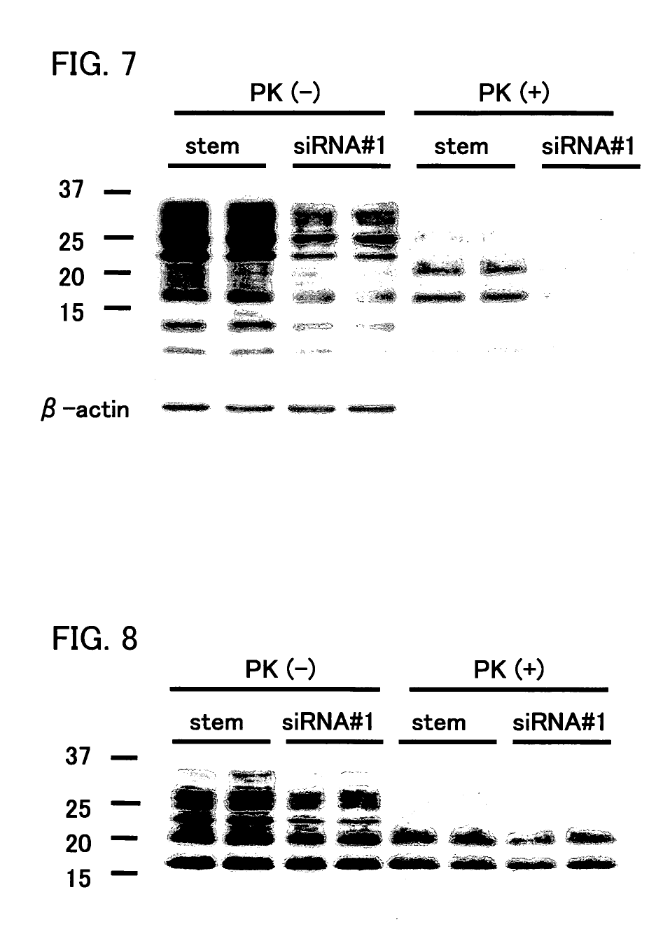

The prion protein specific siRNA (No.1) expression vector was transfected into Fukuoka-1 prion strain infected cells. After 96-hour incubation, detection of the prion protein was carried out by the western blotting method. Results are shown in FIG. 7. In FIG. 7, stem indicates the control vector, and PK(−) and PH(+) indicate whether the protenase K treatment was carried out or not. The numerical values (15, 20, 25, and 37) on the left side indicate molecular weights of proteins. β-actin was used as an internal control. As clearly seen from FIG. 7, the production of the prion protein was inhibited both in PK(−) and PK(+), compared with the control. This shows that it is possible to inhibit the abnormal prion protein accumulation even in the Fukuoka-1 prion strain that has a different property from the Chandler prion strain.

### EXAMPLE 5

### Prion Protein Inhibition Effect in 22L Prion Strain Infected Cells

The prion protein specific siRNA (No.1) expression vector was transfected into a 22L prion strain infected cells. After 96-hour incubation, detection of the prion protein was carried out by the western blotting method.

Results are shown in FIG. 8. In FIG. 8, stem indicates the control vector, whereas PK(−) and PK(+) indicate whether or not the protenase K treatment was carried out or not. The numerical values (15, 20, 25, and 37) on the left side indicate molecular weights of proteins. From FIG. 8, it is clear that the production of the prion protein was inhibited both in PK(−) and PK(+), compared with the control. However, the inhibition was not so high as the inhibition effects observed in the Chandler prion strain infected cells and the Fukuoka-1 prion strain infected cells.

From those results, it was proved that the abnormal prion protein inhibition method of the present invention is effective in inhibiting the abnormal prion protein accumulation in plural kinds of prion strains, even though different prion strains show different sensitivities. Hence, the abnormal prion protein inhibition method of the present invention is quite useful in clinical application.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method of inhibiting accumulation of an abnormal prion protein by inhibiting expression of a normal prion protein in prion-infected cells.

2. The abnormal prion protein inhibition method as set forth in claim 1, wherein:
the expression of the normal prion protein is inhibited by using a prion protein specific siRNA.

3. The abnormal prion protein inhibition method as set forth in claim 2, comprising the step of:
introducing the prion protein specific siRNA or an siRNA expression vectors into the prion-infected cells, the siRNA expression vector having a prion protein specific sequence.

4. The abnormal prion protein inhibition method as set forth in claim 3, wherein:
the siRNA expression vector is a virus vector.

5. The abnormal prion protein inhibition method as set forth in claim 4, wherein:
the virus vector is an adenovirus vector, a herpesvirus vector, or a retrovirus vector.

6. The abnormal prion protein inhibition method as set forth in claim 1, wherein:
the prion proteins are human prion proteins, domestic animal prion proteins, or laboratory animal prion proteins.

7. A method of preventing and/or treating a prion disease, comprising the step of:
inhibiting accumulation of an abnormal protein in prion-infected cells by inhibiting expression of a normal prion protein in the prion-infected cells.

8. The method as set forth in claim 7, wherein:
the expression of the normal prion protein is inhibited by using a prion protein specific siRNA.

9. The method as set forth in claim 8, comprising the step of:
introducing the prion protein specific siRNA or an siRNA expression vector into the prion-infected cells, the siRNA expression vector having a prion protein specific sequence.

10. The method as set forth in claim 9, wherein:
the siRNA expression vector is a virus vector.

11. The method as set forth in claim 10, wherein:
the virus vector is an adenovirus vector, a herpesvirus vector, or a retrovirus vector.

12. The method as set forth in claim 7, wherein:
the prion proteins are human prion proteins, domestic animal prion proteins, or laboratory animal prion proteins.

13. An siRNA expression vector having a prion protein specific sequence.

14. The siRNA expression vector as set forth in claim 13 being a virus vector:

15. The siRNA expression vector as set forth in claim 14 being an adenovirus vector, a herpesvirus vector, or a retrovirus vector.

16. The siRNA expression vector as set forth in claim 13, wherein:
the prion protein specific sequence is a human prion protein specific sequence, a domestic animal prion protein specific sequence, or a laboratory animal prion protein specific sequence.

17. A medicament for preventing and/or treating a prion disease, the medicament containing a prion protein specific siRNA.

18. The medicament as set forth in claim 17, wherein: the prion protein specific sequence is a human prion protein specific sequence, a domestic animal prion protein specific sequence, or a laboratory animal prion protein specific sequence.

19. A medicament of preventing and/or treating the prion disease, the medicament containing an siRNA expression vector having a prion protein specific sequence.

20. The medicament as set forth in claim 19, wherein:
the vector is a virus vector.

21. The medicament as set forth in claim 20, wherein
the virus vector is an adenovirus vector, a herpesvirus vector, or a retrovirus vector.

22. The medicament as set forth in claim 19, wherein:
the prion protein specific sequence is a human prion protein specific sequence, a domestic animal prion protein specific sequence, or a laboratory animal prion protein specific sequence.

23. A kit comprising:
an siRNA expression vector having a prion protein specific sequence; and
a member for administering the vector.

24. The kit as set forth in claim 23, wherein:
the vector is a virus vector.

25. The kit as set forth in claim 24, wherein:
the virus vector is an adenovirus vector, a herpesvirus vector, or a retrovirus vector.

26. The kit as set forth in claim 23, wherein:
the prion protein specific sequence is a human prion protein specific sequence, a domestic animal prion protein specific sequence, or a laboratory animal prion protein specific sequence.

27. A kit for use in prion research, containing an siRNA expression vector having a prion protein specific sequence.

28. The kit as set forth in claim 27, wherein:
the vector is a virus vector.

29. The kit as set forth in claim 28, wherein:
the virus vector is an adenovirus vector, a herpesvirus vector, or a retrovirus vector.

30. The kit as set forth in claim 27, wherein:
the prion protein specific sequence is a human prion protein specific sequence, a domestic animal prion protein specific sequence, or a laboratory animal prion protein specific sequence.
